(19) United States
(12) Patent Application Publication
(10) Pub. No.: US 2006/0125620 A1
(43) Pub. Date: Jun. 15, 2006

(54) Monitoring and security system and method

(76) Inventors: Timothy D. Smith, Birmingham, MI (US); Anthony Bingham, Clarkston, MI (US)

Correspondence Address: Thomas G. Scavone;Niro, Scavone, Haller & Niro, Suite 4600, 181 W. Madison, Chicago, IL, 60602, US

(21) Appl. No.: 11/234,687

(22) Filed: Sep. 23, 2005

Related U.S. Application Data
(63) Continuation-in-part of application No. 10/944,675, filed on Sep. 17, 2004.

(51) Int. Cl. G08B 29/00; A61B 5/00; G08B 19/00; G08B 1/00; G08B 1/08; G08B 23/00

(52) U.S. Cl. 340/506; 340/573.1; 340/521; 340/531; 340/539.12; 600/300

(57) ABSTRACT

A system and method simplifying, combining and improving vehicle and personal diagnostic monitoring and reporting, automatic vehicle emergency event notification, personal emergency distress notification, vehicle and personal location notification, vehicle and personal threat notification and remote vehicle operation (door locks, lights, horn and other systems). System and method include a device within the vehicle, or carried by an individual, and having a wireless communication capability, providing two way communication of data via a hand-held device or key fob, and a remote call center capable of storing, analyzing and assessing services required and communicating back the results of that assessment. Services would include roadside mechanic assistance, towing, informed Public Safety Answering Point (PSAP) notification (including assessment of severity of emergency) and threat notification.

### RELATED APPLICATIONS

This is a continuation-in-part of U.S. patent application Ser. No. 10/944,675 filed Sep. 17, 2004.

### BACKGROUND OF THE INVENTION

1. Field of the Invention

The present invention generally relates to personal monitoring equipment capable of automatic emergency event notification, personal emergency distress notification, vehicle and personal location notification, and personal threat notification.

2. Description of the Related Art

Motor vehicles contain complex mechanical systems that are monitored and regulated by on-board computer systems often known as electronic vehicle control units (VCUs).

Such VCUs monitor various vehicle systems including powertrain performance, crash sensors, air bag deployment and the like, and store codes associated with behaviors that can subsequently be used to diagnose vehicular systems issues.

These codes are generally used to trigger a message on the vehicle console, for example: “check engine,” “service engine soon,” or “change oil.” The service center, once engaged, will then attach diagnostic equipment that deciphers these codes providing more detail regarding said issues and their solutions.

A remote diagnostics service in addition to emergency service, remote vehicle access, directions and other services are now an available option on “OnStar” equipped vehicles. The emergency and location based services, although extremely useful to an individual person regardless of their proximity to their vehicle, are limited to the vehicle and its location, and are unavailable when the user is outside or away from the automobile.

There are devices that fulfill the emergency and location based services, such as products and services from “Life Alert” and “Wherify,” but these products are unable to communicate with the user's vehicle and notify the service of an in-vehicle emergency event such as a crash or an airbag deployment and they certainly do not provide other vehicle diagnostic monitoring and reporting functions.

There also exist personal monitoring devices that may be manually actuated by an individual to provide a request for assistance or distress signal via a wireless communications network. However, these devices may not operate effectively in circumstances where the user is incapacitated.

There also exist wireless communication devices and protocols both in and outside of the vehicle. Mobile, cell or PCS technology phone systems abound providing long distance wireless communication capabilities. In addition there are other wireless communication protocols that provide for short range communications using radio frequency protocols. One such example being the “Bluetooth” protocol.

In vehicle use of the “Bluetooth” protocol is at present being used to provide “hands-free” use of mobile phones or communication with portable music or “MP3” devices.

As a result, an individual wanting services and devices that provide personal diagnostic monitoring and reporting, emergency event notification, personal emergency distress notification, vehicle and personal location notification, vehicle and personal threat notification and remote vehicle operation must currently purchase a number of separate devices and services to accomplish this. In general, however, a user doesn't want to carry or pay for multiple devices or services. A user wants a small personal device that comfortably fits into their pocket or otherwise on the person that integrates their wants and needs. Automotive OEM's want product offerings to compete with GM but do not want to take on the burden and expense of building an “OnStar” type infrastructure and do not want to license a GM branded product.

### SUMMARY OF THE INVENTION

In view of the foregoing and other problems of the conventional methods and structures, an object of the present invention is to provide a method and structure to combine prior and new art into an integrated and affordable personal system and service available to individuals in need of an automatic personal notification device and to all automobile owners and their families.

Accordingly, the present invention is directed to an interactive monitoring and security system comprising a central station, a plurality of portable remote user stations and a plurality of system equipped vehicles. The central station has capabilities for both wireless communication with each of the user stations and for processing data received from the user stations. The remote user stations, in turn, are each capable of wireless communication with the central station and with one of the vehicles. Finally, each vehicle in the system includes a microprocessor control unit that monitors various vehicle systems. The control unit may also include a wireless communications interface or, alternatively, is wired to the interface to both transmit and receive data to and from the associated user station.

The invention is also directed to a personal monitoring system comprising a central station and a plurality of remote user stations in wireless communication with the central stations. Each remote user station includes a control unit which monitors one or more of an individual's personal physical parameters (heart rate, pulse, blood pressure, etc.), and a transmitter which automatically issues a signal to the central station when the monitored physical parameter meets or exceeds some predetermined value. The remote user stations also employ an alarm which is omitted when the predetermined physical parameter value is met. The issued signal contains identification, voice communication, and/or location data for the individual associated with the transmitting remote user station. The remote user station may also employ a receiver for signals transmitted to it from the central station.

The invention is also directed to the method of operating such monitoring and security systems.

In a first aspect of the present invention, therefore, the remote user station will establish secure communications with the vehicle, when in close proximity to the vehicle. It will receive encoded information transmitted by the vehicle on-board control unit and route this data to a central station which will collect, process and or act upon that information. The transmission between the remote user station and on-board unit will preferably utilize “Bluetooth” (or other wireless/RF technology, as appropriate). Transmission between the remote user station and the central station will utilize an appropriate mobile, cellular or PCS communication protocol.

The central station will be able to establish secure communications with the remote user station which will receive encoded information and route this data to the vehicle on-board unit, when in close proximity to the vehicle, which will act upon this code as necessary.

The user will also be able to send a call to the central station with the remote station device regardless of the user's proximity to the vehicle. The device will allow the call center to determine its location (GPS tracking) and provide required assistance utilizing two-way voice communication.

The user will be able to record a brief message that can be stored and played back at user's convenience.

This system and method benefits the vehicle manufacturer and service provider by enhancing customer loyalty and providing timely emergency response and feedback to solve engineering and manufacturing problems. The system and method benefits the users by providing a small personal device that comfortably fits into their pocket that integrates their wants and needs, within and away from their vehicle.

### BRIEF DESCRIPTION OF THE DRAWINGS

The novel features which are characteristic of the present invention are set forth in the appended claims. However, the invention's preferred embodiments, together with further objects and attendant advantages, will be best understood by reference to the following detailed description taken in connection with the accompanying drawings in which:

FIG. 1 is a schematic representation of one preferred embodiment of the interactive monitoring and security system of the present invention;

FIG. 2 is a perspective view of one preferred embodiment of the portable remote user station made in accordance with the present invention; and

FIG. 3 is also a schematic representation of another preferred embodiment of the interactive monitoring and security system of the present invention.

### DETAILED DESCRIPTION OF THE PREFERRED EMBODIMENTS

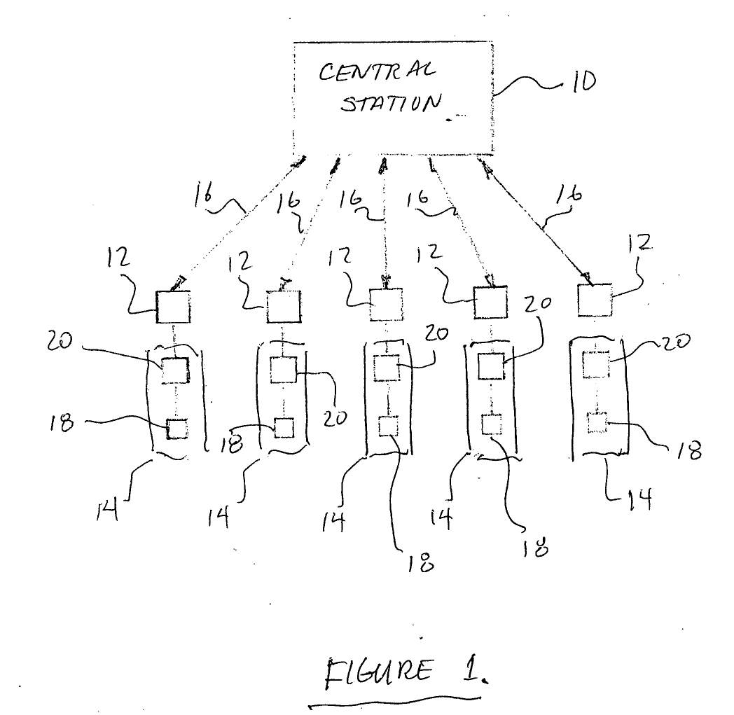

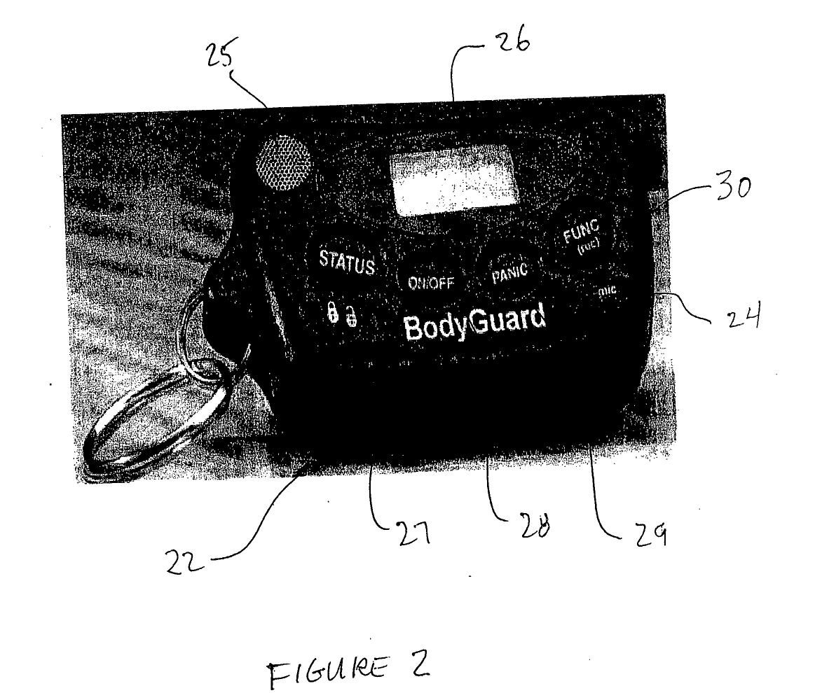

Referring now to the drawings, and more particularly to FIGS. 1-2, there are shown preferred embodiments of the method and structures according to the present invention. As illustrated in FIG. 1, one preferred embodiment of the invention comprises a central station 10, a plurality of portable remote user stations 12, and a plurality of suitably equipped vehicles 14.

The central station 10 includes a call center to receive audio communications and data transmitted from each of the remote stations 12. The central station also includes data processing capabilities for analyzing various data received from the remote stations 12. All of the audio and data transmissions between the central and remote stations is effected over convention wireless communications networks, illustrated schematically as lines 16. The central station 10 can also locate each of the remote user stations 12 via GPS.

The remote stations 12 may take many forms. These devices are most preferably hand-held and completely transportable on the user's person. Two preferred embodiments comprise a cell phone and a key fob. The remote stations 12 can wirelessly communicate with the central station 10 and, using Bluetooth or other radio frequency technology, each can wirelessly communicate with one of the vehicles 14 in the system. The remote stations also include GPS functionality and a storage capacity for retaining vehicle data and audio messages inputted by the user. Preferably, the portable remote stations 12 also have one or more function buttons and a display for text messaging.

The vehicles 14 employed in the preferred embodiments of the invention include a microprocessor control unit 18 which is operatively associated with various vehicle systems and functions. These systems and functions may include engine performance parameters, fuel and oil levels, airbag readiness and deployment, tire pressure, odometer and speedometer readings, and many others. The vehicle systems and functions are monitored by control unit 18 which can store relevant vehicle data and transmit this data to the remote user stations through an integral or separate communications interface 20. A preferred from of communications interface 20 is a conventional Bluetooth transmitter and receiver.

FIG. 2 illustrates one preferred portable user station in the form of a key fob 22. The fob 22 may include a microphone 24, a speaker 25, a visual display 26, and a variety of function buttons 27-30. These function buttons may establish a communication link with the central station, send out an emergency signal, lock and unlock the vehicle and perform many other functions well known in the art.

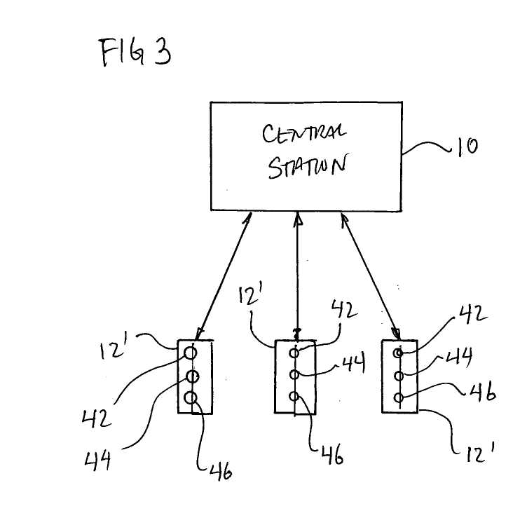

With reference now to FIG. 3, another embodiment of the invention directed to a personal monitoring system is shown. This embodiment makes use of the same central station 10 and a plurality of remote user stations 12. Remote user stations 12 may have all of the functionality described above in connection with remote user stations 12, or may be more suitably constructed for use only as a personal monitoring device. Remote user station 12 is portable, battery powered and designed for transport with the person using it. For example, it may be in the form of a pendant, bracelet, or pocket carried. The remote user station 12′ includes a control unit 42 that monitors a particular physical parameter of interest. Examples of such parameters include respiration, heart rate, blood pressure, blood sugar level, body temperature, ambulatory function, etc. The remote user station 12 also includes a wireless transmitter 44 which automatically issues a signal to the central station whenever the monitored physical parameter of the individual meets a predetermined value. The issued signal includes identification data and/or location data for the individual associated with the transmitting remote user station; it may also provide data relating to the particular monitored physical condition. In addition, remote user station 12 may also employ an audible, visible or tactile alarm which is emitted when the predetermined physical parameter is met. Finally, the remote user station 12 may also include a receiver 46 for communications transmitted to it from the central station. The remote user station controller, transmitter, alarm, and receiver may all comprise conventional components well known to those of skill in the art.

It will be appreciated by those skilled in the art that various changes and modifications can be made to the illustrated embodiments without departing from the spirit of the present invention. All such modifications and changes are intended to be covered by the appended claims.

What is claimed:

1. An interactive monitoring and security system comprising:
a central communication and data processing station;
a plurality of portable remote user stations in wireless communication with the central station; and
each remote user station having a control unit that monitors at least one physical parameter of an individual associated with the remote user station and a transmitter which automatically issues a signal from the remote user station to the central station in response to the at least one physical parameter meets a predetermined value.

2. The system of claim 1 wherein the transmitted signal includes identification data for the individual associated with the remote user station issuing the signal transmission.

3. The system of claim 1 wherein the transmitted signal includes location data for the individual associated with the remote user station issuing the transmission signal.

4. The system of claim 1 wherein the at least one physical parameter is respiratory function.

5. The system of claim 1 wherein the at least one physical parameter is a cardiac function.

6. The system of claim 1 wherein the at least one physical parameter is any one or more of respiration, heart rate, blood pressure, body temperature, blood sugar level or ambulatory function.

7. The system of claim 1 wherein the remote user station also includes an audible, visual or tactile alarm which is emitted automatically in response to the at least one physical parameter meeting said predetermined value.
